(19) United States
(12) Patent Application Publication
(10) Pub. No.: US 2026/0338805 A1
(43) Pub. Date: Oct. 29, 2026

(54) METHOD FOR ANALYZING BLOOD COAGULATION ABILITY OF BLOOD SPECIMEN

(71) Applicants: SEKISUI MEDICAL CO., LTD., Tokyo (JP); PUBLIC UNIVERSITY CORPORATION NARA MEDICAL UNIVERSITY, Kashihara-shi (JP)

(72) Inventors: Toshiki KAWABE, Chuo-ku (JP); Kengo ONISHI, Chuo-ku (JP); Keiji NOGAMI, Kashihara-shi (JP); Kenichi OGIWARA, Kashihara-shi (JP); Naruto SHIMONISHI, Kashihara-shi (JP)

(73) Assignees: SEKISUI MEDICAL CO., LTD., Tokyo (JP); PUBLIC UNIVERSITY CORPORATION NARA MEDICAL UNIVERSITY, Kashihara-shi (JP)

(21) Appl. No.: 19/142,489

(22) PCT Filed: Dec. 28, 2023
(86) PCT No.: PCT/JP2023/047187

§ 371 Date: Jun. 23, 2025

(30) Foreign Application Priority Data
Dec. 28, 2022 (JP) 2022-212338

(51) Int. Cl. G01N 33/86

(52) U.S. Cl. CPC G01N 33/86; G01N 2333/755

(57) ABSTRACT

A method for analyzing a blood coagulation ability of a blood specimen. The method includes: 1) acquiring a first derivative V(i) of a coagulation reaction curve of a subject blood specimen, where i denotes the number of measurement points or time; and 2) the following a) or b): a) determining pₖ, which is a minimum of a point at which V(i) reaches Xₖ or higher when V(i) reaches a maximum Vmax or therebefore, and qₖ, which is a maximum of a point at which V(i) reaches Xₖ or higher when V(i) reaches the maximum Vmax or thereafter, where k represents a series of integers from 1 to n, n is an integer greater than or equal to two, Xₖ is Vmax×Sₖ%, and 0<Sₖ≤100; b) determining a relativized maximum of V(i).

### TECHNICAL FIELD

The present invention relates to a method for analyzing a blood coagulation ability of a blood specimen.

### BACKGROUND ART

Hemophilia A is a hemorrhagic disease caused by deficiency or dysfunction of coagulation factor VIII (FVIII). Hemophilia A is classified into congenital hemophilia A with congenital deficiency of FVIII, and acquired hemophilia A caused by autoantibody (inhibitor) to FVIII. Some patients with congenital hemophilia A have inhibitors. Hemophilia A is classified into mild, moderate, and severe depending on the FVIII activity level in the blood. Specimens derived from patients with hemophilia A have a prolonged blood coagulation time in a blood coagulation test depending on the severity.

Emicizumab, which is a hemophilia A treatment, is a recombinant humanized bispecific antibody which can promote the blood coagulation reaction. Emicizumab can bind activated coagulation factor IX (FIXa) at one antigen-binding site, bind coagulation factor X (FX) at the other antigen-binding site, and substitute for the cofactor function of activated coagulation factor VIII (FVIIIa), thereby allowing the blood coagulation reaction to be promoted.

Emicizumab is regularly administered to patients with hemophilia A for the purpose of suppressing the bleeding tendency of patients with hemophilia A. The administered emicizumab remains in the blood for a certain period, during which a prolonged blood coagulation time, i.e., a typical characteristic of coagulation abnormality, is not observed in a blood coagulation test of a blood specimen obtained by drawing blood from a patient with hemophilia A to whom emicizumab has been administered. Accordingly, the result shows as if there were no coagulation abnormality. Meanwhile, there is a possibility that reduction in prolonged coagulation time of a patient with hemophilia A (to the reference range upper limit or less) by emicizumab poses a significant danger to the patient. For example, in a case where a patient with hemophilia A undergoes emergency transport and a surgical operation due to a traffic accident or the like, and no prolonged coagulation time (activated partial thromboplastin time: APTT) is observed in a typical preoperative blood coagulation test, a doctor who does not know that the patient has hemophilia A cannot find the patient's reduction in coagulation ability and cannot administer an appropriate treatment. Accordingly, a risk of serious intraoperative bleeding occurs.

Patent Literature 1 describes a method for evaluating a blood coagulation reaction with a substance having a FVIII-substituting activity, by adding a blood coagulation initiation reagent containing activated coagulation factor XI to a blood specimen, and measuring the amount of generated thrombin. Patent Literatures 2 and 3 describe a method for evaluating a coagulation ability of a blood specimen, the method including: acquiring a parameter about the derivative of a coagulation waveform about the blood specimen to which a substance having a FVIII-substituting activity has been administered; acquiring a FVIII activity as a value indicating the coagulation ability from the parameter; and evaluating the coagulation ability of the blood specimen, based on the acquired FVIII activity.

### CITATION LIST

### Patent Literature

Patent Literature 1: WO 2014/050926
Patent Literature 2: WO 2016/170944
Patent Literature 3: JP-A-2017-106925

### SUMMARY OF INVENTION

### Technical Problem

The present invention provides a method for analyzing a blood coagulation ability of a blood specimen, the method allowing detecting the blood specimen to which a drug having FVIII-substituting activity has been administered.

### Solution to Problem

The present invention provides the following. [1] A method for analyzing a blood coagulation ability of a blood specimen, the method comprising:
1) acquiring a first derivative V(i) of a coagulation reaction curve of a subject blood specimen in which presence or absence of a substance having coagulation factor VIII-substituting activity is required to be detected, where i denotes the number of measurement points or time; and
2) the following a) or b):
   a) determining pₖ, which is a minimum of a point at which V(i) reaches Xₖ or higher when V(i) reaches a maximum Vmax or therebefore, and qₖ, which is a maximum of a point at which V(i) reaches Xₖ or higher when V(i) reaches the maximum Vmax or thereafter, where k represents a series of integers from 1 to n, n is an integer greater than or equal to two, Xₖ is Vmax×Sₖ%, and 0<Sₖ≤100;
   b) determining a relativized maximum of V(i).
[2] The method according to [1], wherein the subject blood specimen is a blood specimen with no prolonged activated partial thromboplastin time (APTT).
[3] The method according to [1] or [2], wherein the n is an integer from 5 to 50.
[4] The method according to any one of [1] to [3], further comprising calculating a parameter for analyzing the blood coagulation ability of the subject blood specimen, based on the pₖ or qₖ.
[5] The method according to [4],
   wherein the parameter is at least one selected from the group consisting of a pre-post average difference, CV, qp ratio, VB(Sₖ), kurtosis vAB(Sₖ), rate over time vTB(Sₖ), vAB(Sₖ)×VmaxT, vAB(Sₖ)×APTT, parameters about relative values of pₖ and qₖ, and their standard deviation indices (SDI), where
   the pre-post average difference represents (average of qₐ to q_{b}—average of pₐ to p_{b})/(average of pₐ to p_{b} and qₐ to q_{b}) (where a and b are integers selected from the group consisting of 1 to n, n is as defined above, and a<b),
   the CV represents a coefficient of variation of pₐ to p_{b} and qₐ to q_{b} (where a and b are integers selected from the group consisting of 1 to n, n is as defined above, and a<b),
   the qp ratio represents qₖ/pₖ,
   the vB(Sₖ) represents an interval from pₖ to qₖ,
   the VmaxT satisfies V(VmaxT)=Vmax,
   the APTT is an activated partial thromboplastin time (APTT) of the subject blood specimen,
   the vAB(Sₖ)=vH(Sₖ)/vB(Sₖ), and
   the vTB(Sₖ)=vB(Sₖ)/VmaxT,
   where
   the vH(Sₖ) is of the following equation,

$$vH ( Sk ) = \frac{{\sum }_{i = pk}^{qk} ( i \times V ( i ) )}{{\sum }_{i = pk}^{qk} i}$$

[6] The method according to [5],
   wherein the parameters about the relative values of the pₖ and qₖ are slopes of linear regression lines of a plot from pₐ to p_{b} of the subject blood specimen and a plot from q_{c} to q_{d} of the subject blood specimen with respect to pₐ to p_{b} of a reference specimen and q_{c} to qₐ of a reference specimen, respectively (where a, b, c, and d are integers each independently selected from the group consisting of 1 to n, a<b, c<d, and n is as defined above), and
   the reference specimen is a blood specimen to which no drug having FVIII-substituting activity has been administered.
[7] The method according to any one of [1] to [3], further comprising calculating a parameter for analyzing the blood coagulation ability of the subject blood specimen, based on the relativized maximum of V(i).
[8] The method according to [7],
   wherein the parameter is at least one selected from the group consisting of a corrected Vmax, the corrected Vmax×the VmaxT, the corrected Vmax×the APTT, and their standard deviation indices (SDI),
   the corrected Vmax is the relativized maximum of V(i),
   the VmaxT satisfies V(VmaxT)=Vmax, and
   the APTT is an activated partial thromboplastin time (APTT) of the subject blood specimen.
[9] The method according to any one of [4] to [8], further comprising detecting the presence or absence of a substance having coagulation factor VIII-substituting activity in the subject blood specimen, based on the parameter.
[10] The method according to [9], further comprising outputting a result of the detecting the presence or absence of a substance having coagulation factor VIII-substituting activity.
[11] The method according to any one of [1] to [10], further comprising:
   measuring the activated partial thromboplastin time (APTT) of a blood specimen; and
   selecting the blood specimen with no prolonged APTT, as the subject blood specimen in which the presence or absence of a substance having coagulation factor VIII-substituting activity is required to be detected.
[12] The method according to any one of [1] to [11], wherein the substance having coagulation factor VIII-substituting activity is a bispecific antibody substituting for a cofactor function of coagulation factor VIII.
[13] The method according to any one of [1] to [12], wherein the substance having coagulation factor VIII-substituting activity is emicizumab.
[14] A program for implementing the method according to any one of [1] to [13].
[15] A device for implementing the method according to any one of [1] to [13].
[16] A system for implementing the method according to any one of [1] to [13], the system comprising: a program for implementing the method according to any one of [1] to [13]; and a device controlled by the program.
[17] The device according to [15] or the system according to [16], further comprising an output unit for outputting a result of analysis of the blood coagulation ability of the blood specimen by the method according to any one of [1] to [131].

### Advantageous Effect of Invention

The present invention can detect that a test specimen is a blood specimen to which a drug having FVIII-substituting activity, such as emicizumab, has been administered, among blood specimens in which no coagulation abnormality can be detected by conventional APTT measurement, using measured data (coagulation reaction data) of a coagulation reaction acquired during APTT measurement.

### BRIEF DESCRIPTION OF DRAWINGS

FIG. 1 illustrates an example of a coagulation reaction curve.

FIG. 2 is a conceptual diagram for describing Xₖ, pₖ, and qₖ.

FIG. 3A illustrates an example of a blood coagulation analyzing process by a method of the present invention.

FIG. 3B is a conceptual diagram illustrating a configuration of a device for performing a method for analyzing a blood coagulation ability by the present invention.

FIG. 4 illustrates APTT of a specimen group used in Example. NE: control specimen group, E1: specimen derived from a patient with hemophilia A to whom emicizumab has been administered. E2: specimen which contains an inhibitor and has been derived from a patient with hemophilia A to whom emicizumab has been administered.

FIG. 5 illustrates coagulation reaction curves R(i) (upper) and first derivative curves V(i) (lower) of NE, E1, and E2.

FIG. 6 illustrates corrected reaction rate curves of NE, E1, and E2.

FIG. 7 illustrates A: a plot of Vmax against APTT of each specimen, and B: distributions of Vmax of each specimen group.

FIG. 8 illustrates A to D: plots of Vmax against APTT of NE, E1, E2, and all the specimens, and E: a distribution of VmaxT of each specimen group.

FIG. 9 illustrates A: a plot of corrected Vmax against APTT of each specimen, where dotted line indicates a threshold line, and B: a distribution of corrected Vmax of each specimen group.

FIG. 10 illustrates A: a plot of [corrected Vmax×VmaxT] against APTT of each specimen, and B: a distribution of [corrected Vmax×VmaxT] of each specimen group.

FIG. 11 illustrates A: a plot of [corrected Vmax×APTT] against APTT of each specimen, and B: a distribution of [corrected Vmax×APTT] of each specimen group.

FIG. 12 illustrates A: a plot of vTB(30) (=vB(30)/VmaxT) against APTT of each specimen, and B: a distribution of vTB(30) of each specimen group.

FIG. 13 illustrates A: a plot of vAB(10) (=vH(10)/vB(10)) against APTT of each specimen, where dotted line indicates a threshold line, and B: a distribution of vAB(10) of each specimen group.

FIG. 14 illustrates A: a plot of [vAB(10)×VmaxT] against APTT of each specimen, B: a distribution of [vAB(10)×VmaxT] of each specimen group, C: a plot of [vAB(10)×APTT] against APTT of each specimen, and D: a distribution of [vAB(10)×APTT] of each specimen group.

FIG. 15 illustrates A: a plot of a qp ratio (q₈/p₈) to APTT of each specimen, and B: a distribution of the pq ratio of each specimen group.

FIG. 16 illustrates A: a plot of q₆−p₆ to APTT of each specimen, where dotted line indicates a threshold line, and B: a distribution of q₆−p₆ of each specimen group.

FIG. 17 illustrates A: a plot of a pre-post average difference against APTT of each specimen, B: a distribution of the pre-post average difference of each specimen group, C: a plot of a lower-side pre-post average difference against APTT of each specimen, D: a distribution of the lower-side pre-post average difference of each specimen group, E: a plot of a middle-side pre-post average difference against APTT of each specimen, and F: a distribution of the middle-side pre-post average difference of each specimen group.

FIG. 18 illustrates A: a plot of all CV against APTT of each specimen, B: a distribution of all CV of each specimen group, C: a plot of lower CV against APTT, and D: a distribution of lower CV of each specimen group.

FIG. 19 illustrates a slope of a regression line of relative pₖ and relative qₖ (k=1 to 20), A and B: in a case where an NE specimen with minimum APTT is employed as a reference specimen (●), a plot of the slope of the regression line against APTT (A), and a distribution of each specimen group (B), and C and D: in a case where the NE specimen with maximum APTT is employed as the reference specimen (●), a plot of the slope of the regression line against APTT (C), and a distribution of each specimen group (D).

FIG. 20 illustrates the slope of the regression line of the relative pₖ and the relative qₖ (k=6 to 20), A and B: in a case where the NE specimen with minimum APTT is employed as the reference specimen (●), a plot of the slope of the regression line against APTT (A), and a distribution of each specimen group (B), and C and D: in a case where the NE specimen with maximum APTT is employed as the reference specimen (●), a plot of the slope of the regression line against APTT (C), and a distribution of each specimen group (D).

### DESCRIPTION OF EMBODIMENTS

All Patent Literatures, Non Patent Literatures, and other publications cited in the present Description are incorporated herein by reference in their entirety.

In a blood coagulation test for measuring the activated partial thromboplastin time (APTT), a predetermined reagent is added to a subject blood specimen, a subsequent blood coagulation reaction is measured, and the blood coagulation time (APTT) is calculated from the coagulation reaction data. To measure the blood coagulation reaction, general means is used; for example, optical means for measuring the scattered light intensity, transmittance, and absorbance, or mechanical means for measuring the viscosity of plasma are used. Typically, the blood coagulation reaction is represented by a coagulation reaction curve which indicates the temporal change in the amount of coagulation reaction. In the following Description, the blood specimen, blood coagulation reaction, and blood coagulation time are sometimes simply called the specimen, coagulation reaction, and coagulation time, respectively.

With specimens having a coagulation abnormality factor, APTT is often prolonged in comparison with that of a normal specimen with no coagulation abnormality. The prolongation of APTT is one of major indicators for the coagulation abnormality related to a bleeding risk or a thrombosis risk. With a specimen derived from a patient with hemophilia A, APTT is typically prolonged beyond a reference range (a range allowing a specimen having APTT within this range to be regarded “normal”). Meanwhile, in a case where a substance having FVIII-substituting activity (hereinafter, also called “FVIII-substituting substance”), such as emicizumab, has been administered to a patient with hemophilia A, APTT of a specimen of the patient is not prolonged, and falls within the reference range or is reduced below the reference range. It is difficult to distinguish by APTT measurement whether the test specimen is a specimen derived from a patient with hemophilia A to which the FVIII-substituting substance has been administered or not based on the reference range of APTT.

The present inventors found that even in a case where no prolonged coagulation time is observed by measuring APTT, the blood specimen derived from a patient with hemophilia A to whom emicizumab has been administered exhibits a different progression of the blood coagulation reaction (blood coagulation reaction rate curve) from that of a specimen to which no emicizumab has been administered.

In the present invention, the presence or absence of FVIII-substituting substance in the test specimen is detected based on coagulation reaction data of a test specimen measured during APTT measurement, specifically, a parameter derived from the blood coagulation rate curve obtained by the first derivative of the coagulation reaction curve. In the present invention, among specimens in which no coagulation abnormality is detected by conventional APTT measurement, a specimen to which the FVIII-substituting substance has been administered can be detected.

In the present Description, the FVIII-substituting substance refers to a substance that can substitute for an FVIII cofactor function. The FVIII cofactor function refers to a function of activated FVIII (FVIIIa) forming a complex of activated coagulation factor IX (FIXa) and coagulation factor X (FX). An example of the FVIII-substituting substance may be a bispecific antibody substituting for the FVIII cofactor function (for example, a bispecific antibody simulating the FVIII structure). An example of such a bispecific antibody may be emicizumab. Emicizumab is a recombinant humanized bispecific antibody that binds the activated coagulation factor IX (FIXa) at one antigen-binding site, and binds the coagulation factor X (FX) at the other antigen-binding site, and substitutes for the FVIII cofactor function. Emicizumab is a drug for suppressing the bleeding tendency of congenital and acquired hemophilia A. Furthermore, next-generation FIXa/FX bispecific antibodies (Mim8, NXT007 and the like) having an action mechanism similar to that of emicizumab have also been developed.

### [Method for Analyzing Blood Coagulation Ability of Blood Specimen]

The method for analyzing a blood coagulation ability of a blood specimen in the present invention (hereinafter also called the method of the present invention) can detect the difference between the coagulation abilities of blood specimens which cannot be discriminated based on the coagulation time (APTT), as the difference in progression of the coagulation reaction (coagulation reaction rate curve). In the method of the present invention, based on the first derivative of the coagulation reaction curve of the test specimen, a parameter for analyzing the blood coagulation ability of the test specimen is acquired.

The method of the present invention is described below.

### 1. Coagulation Reaction Measurement

Preferably, the method of the present invention uses, as the test specimen, the plasma of a subject drawn for a coagulation test. An anticoagulant typically used for a coagulation test may be added to the specimen. For example, blood is drawn using a blood collection tube which contains sodium citrate, and is subsequently centrifugally separated, thus obtaining plasma.

The coagulation reaction curve of a test specimen used for the method of the present invention is calculated from coagulation reaction data acquired in accordance with procedures for measuring the coagulation reaction in typical APTT measurement. Specifically, in coagulation reaction measurement, an APTT measurement reagent is added to the test specimen, and the blood coagulation reaction is started. The coagulation reaction in a mixture containing the reagent and the test specimen is measured. APTT measurement reagents are commercially available (for example, APTT reagent Coagpia APTT-N, and Coagpia APTT-N calcium chloride liquid, which are manufactured by Sekisui Medical Co., Ltd.). To measure the coagulation reaction, general means is used; for example, optical means for measuring the scattered light intensity, transmittance, absorbance or the like, or mechanical means for measuring the viscosity of blood are used. In the following present Description, the method of the present invention is described using, as an example, coagulation reaction measurement based on the scattered light intensity.

The reaction initiation time point of the coagulation reaction can typically be defined as a time point at which the reagent is mixed into the specimen and the coagulation reaction is started. Alternatively, other timing may be defined as a reaction initiation time point. The duration of measuring the coagulation reaction can range, for example, from several tens of seconds to about seven minutes from the time point of mixing the specimen and the reagent. The measurement time may be a freely defined fixed value, or may be up to a time point at which the end of the coagulation reaction of each specimen is detected. During the measurement time, measurement of the progress of the coagulation reaction (measurement of the scattered light intensity) can be repeatedly performed at predetermined intervals. For example, measurement may be performed at intervals of 0.1 seconds. The temperature of the mixture during measurement is under a typical condition, for example, between 30 °C and 40 °C, inclusive, preferably, between 35 °C and 39 °C, inclusive. Various conditions of measurement can be set in conformity with the test specimen, reagent, measurement means and the like as appropriate.

A series of operations in the coagulation reaction measurement described above can be performed using an automatic analysis device. An example of the automatic analysis device may be a blood coagulation automatic analysis device CP3000 (manufactured by Sekisui Medical Co., Ltd). Alternatively, some operations may be manually performed. For example, preparation of the test specimen can be performed by a person, and operations thereafter can be performed by the automatic analysis device.

### 2. Acquisition of Coagulation Reaction Curve and First Derivative V(i)

A coagulation reaction curve R(i) is acquired from the measured coagulation reaction data. Here, “i” denotes the number of measurement points or time from the coagulation reaction initiation (also simply called the number of measurement points, and time, respectively). For example, in the case where a measurement (photometry) interval is 0.1 seconds, it is represented that time=0.1×the number of measurement points. That is, R(i) may be a function of the number of measurement points, or a function of time. Typically, the coagulation reaction curve R(i) is obtained by applying a denoising or smoothing process to measurements of the coagulation reaction measurement by typical means, or applying zero adjustment for adjusting the initial value or curve relativizing as required.

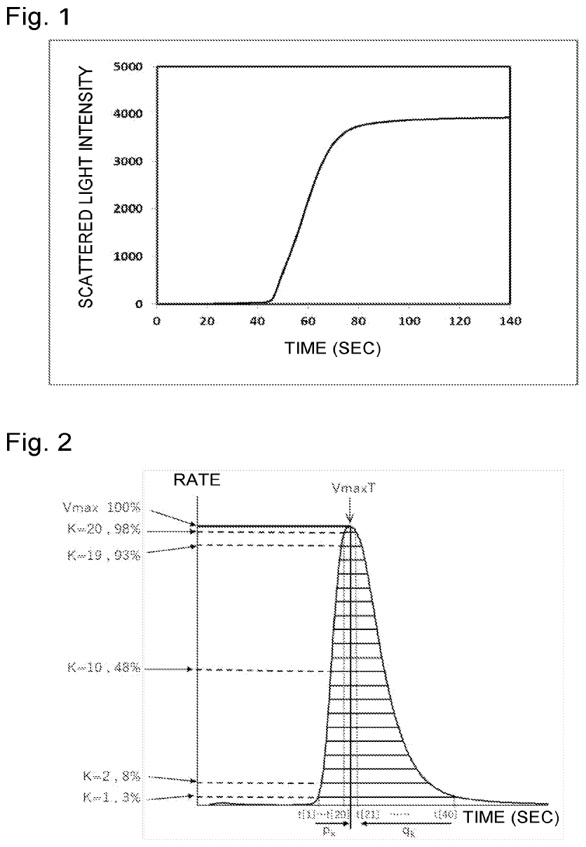

FIG. 1 illustrates an example of the coagulation reaction curve. The abscissa axis of FIG. 1 indicates time, and the ordinate axis indicates the coagulation reaction (scattered light intensity), which are adjusted such that the scattered light intensity at the measurement initiation time point is zero. The coagulation reaction of the mixture proceeds over time. Thus, the scattered light intensity increases. The coagulation reaction curve based on the scattered light intensity as illustrated in FIG. 1 is typically sigmoidal.

From the R(i), its first derivative V(i) is acquired. The differential process for obtaining V(i) from R(i) can be executed by any method, and can be performed by, for example, calculating the intra-interval average slope. In the intra-interval average slope calculation, a predetermined number of measurement points before and after each measurement point i, for example, 2K+1 measurement points from i−K to i+K can be used. Here, K is any integer. For example, in a case where K is two, (i−2)−, (i−1)−, i−, (i+1)−, and (i+2)-th five measurement points can be used. The average slope means a slope when these measurement points are linearly approximated. A usual method, such as a least-squares method, can be used as the computation method for the linear approximation. The average slope of these measurement points can be assumed as the first derivative V(i) of R(i) at the measurement point i. Alternatively, the first derivative of the relative value of R(i) may be acquired as V(i). For example, conversion may be performed such that the maximum of R(i) is a predetermined value, for example, 100, and the first derivative may be obtained, thus acquiring V(i). The first derivative V(i) constitutes a curve representing the rate of the coagulation reaction, or the reaction progress rate.

The acquisition of R(i) and V(i) about the test specimen may be performed in parallel with the measurement of the coagulation reaction of the test specimen, or performed after the measurement of the coagulation reaction is finished. Preferably, the measurement of the coagulation reaction of the test specimen is performed until the coagulation reaction is finished. The end of the coagulation reaction can be determined based on any reference, such as a time point at which R(i) reaches a plateau, after V(i) reaches the maximum, a time point at which it reduces to 0 or a certain value (for example, S % of the maximum or less). Alternatively, as long as the measurements necessary for obtaining an after-mentioned coagulation time (APTT) and a parameter for analyzing the blood coagulation ability of the test specimen are obtained, the coagulation reaction measurement may be ended at a timing before the coagulation reaction is finished.

As described above, each of R(i) and V(i) in the present Description may be a function of the number of measurement points, or a function of time. The after-mentioned parameter for analyzing the blood coagulation ability of the test specimen may be data with reference to the number of measurement points, or data with reference to time. In the following present Description, R(i) and V(i) are sometimes simply abbreviated as R and V, respectively.

### 3. APTT Calculation

The coagulation time (APTT) of the test specimen can be calculated from the coagulation reaction data acquired by the coagulation reaction measurement. The method of calculating APTT is not specifically limited. For example, APTT can be calculated by any method based on the R(i) or V(i). Examples of the APTT calculating method include: a method of calculating, as the coagulation time, a time point at which R(i) reaches N % of R(E) representing the reaction R at the coagulation reaction end point E (percent detection method); a method of calculating, as the coagulation time, a time point at which V(i) reaches the maximum Vmax or N % thereof; a method of calculating the coagulation time based on the temporal change in the accumulated ratio of R(i) in a minute time period (see JP-A-H6-249855, and WO 2021/132552); a method of calculating the coagulation time based on the weighted average time of V(i) (see WO 2021/177452); and a method of calculating, as the coagulation time, a time point after V(i) reaches the maximum Vmax and then R(i) reaches N % of R(Te) where the timing reaching the predetermined value is assumed as the calculation starting point Te (see WO 2021/206107).

### 4. Acquisition of Parameter for Analyzing Blood Coagulation Ability

4.1) Test Specimen in which Parameter should be Acquired

Next, in the method of the present invention, the parameter for analyzing the blood coagulation ability of the test specimen is acquired based on the V(i). In the method of the present invention, the parameter may be acquired for the test specimen in which the presence or absence of the FVIII-substituting substance is required to be detected. Typically, the test specimen in which the presence or absence of the FVIII-substituting substance is required to be detected is a specimen with no prolonged APTT. Without the need to check for the presence or absence of the FVIII-substituting substance, the specimen with prolonged APTT is estimated as a specimen with a coagulation abnormality (that is, a high bleeding risk or thrombosis risk). Accordingly, application of the method of the present invention is not necessarily required.

In one embodiment of the method of the present invention, APTT is calculated based on the coagulation reaction data obtained by measuring any test specimen, and subsequently, a specimen with no prolonged APTT is selected as a test specimen in which the presence or absence of the FVIII-substituting substance is required to be detected. About the selected test specimen, V(i) calculated from the coagulation reaction data obtained by measuring the coagulation reaction can be acquired. The V(i) may be calculated together with the R(i) and the like during APTT measurement, or V(i) may be obtained by measuring APTT, selecting a test specimen with no prolonged APTT to which the method of the present invention should be applied, and then performing calculation based on R(i) of the selected test specimen. In another embodiment of the method of the present invention, from existing coagulation reaction data, a specimen which has been already identified to have no prolonged APTT is selected as a test specimen in which the presence or absence of the FVIII-substituting substance is required to be detected. From the existing coagulation reaction data, V(i) can be acquired.

### 4.2) Parameter Based on Peak Shape of V

In one embodiment, the parameter for analyzing the blood coagulation ability of the test specimen is a parameter in which the progression of the coagulation reaction is reflected, that is, a parameter based on the peak shape of V. In the acquisition of the parameter, first, a minimum pₖ and a maximum qₖ of the number of measurement points or time at which V reaches a preset setting value Xₖ or greater (that is, satisfies V(i)≥Xₖ) are determined. Typically, V has a peak shape having the maximum Vmax as a peak top. Accordingly, pₖ and qₖ can be present at a time point when V reaches Vmax or therebefore or thereafter. That is, V(pₖ−D)<Xₖ≤V(pₖ), and V(qₖ)≥Xₖ>V(qₖ+D). Here, D denotes a measurement interval for coagulation reaction data, D=1 in a case where pₖ and qₖ are with reference to the number of measurement points, and D=0.1 in a case where pₖ and qₖ are with reference to time, for example, measurement is performed at intervals of 0.1 seconds. In the method of the present invention, Xₖ denotes a variable, and pₖ and qₖ corresponding to Xₖ are determined. For example, in a case where n Xₖ are present as (X₁, . . . , Xₙ) (k denotes a series of integers from 1 to n, and n denotes an integer greater than or equal to two), a point pₖ at which V(i) reaches Vmax or reaches Xₖ therebefore and a point qₖ at which V(i) reaches Vmax or reaches Xₖ thereafter are determined, and n pₖ (p₁, . . . , pₙ) and n qₖ (q₁, . . . , qₙ) are obtained. As described above, pₖ and qₖ may be data with reference to the number of measurement points, or data with reference to time. If the coagulation reaction data includes large noise, a peak of V caused by the noise appears, and pₖ and qₖ are possibly erroneously detected. Erroneous detection of pₖ and qₖ can be prevented by removing noise in the coagulation reaction data before acquisition of V, or by removing a peak of V due to the noise or pₖ and qₖ derived therefrom.

The value of the variable Xₖ may be freely set. Preferably, Xₖ is greater than 0 and less than or equal to Vmax. Preferably, Xₖ can be set with reference to Vmax. For example, Xₖ is defined as Vmax×Sₖ(%), where Sₖ can be greater than 0 and less than or equal to 100, preferably, set in a range from 0.5 to 99. Preferably, Sₖ=a+(k−1)×b, where a>0, b>0, and k is as defined above. When a=1 and b=1, Sₖ=k. The maximum of Sₖ is 100. Note that when Sₖ is 100, Xₖ=Vmax, and pₖ=qₖ. For example, in a case where n Xₖ (X₁, . . . , Xₙ) are set, n Sₖ (S₁, . . . , Sₙ) are set (here, k and n are as described above, and each Sₖ is greater than 0 and less than or equal to 100, preferably, from 0.5 to 99, and more preferably, from 5 to 50). The number of set variables Xₖ is not specifically limited. Preferably, the number ranges from 5 to 50, and more preferably, from 10 to 30, that is, n is an integer ranging, preferably from 5 to 50, and more preferably, from 10 to 30. Preferably, n Sₖ are made up of a series of values gradually changing from their minimum to maximum at regular intervals.

That is, pₖ and qₖ satisfy the following formulae.

V(pₖ−D)<Xₖ≤V(pₖ), and V(qₖ)≥Xₖ>V(qₖ+D),

where D denotes the measurement interval.

Note that
pₖ≤VmaxT≤qₖ, where VmaxT is the number of measurement points or time point which satisfies V(VmaxT)=Vmax.

Xₖ=V max×Sₖ(%),
0<Sₖ 100, preferably, 0.5 Sₖ 99, and more preferably, 5 Sₖ 50.
k denotes an integer ranging from 1 to n,
n denotes an integer ranging, preferably, from 5 to 50, and more preferably, from 10 to 30.

Referring to FIG. 2, Xₖ, pₖ, and qₖ are described. In FIG. 2, the first derivative V of the coagulation reaction curve is plotted against time. The peak top of V is the maximum Vmax (100%), and a time point at which V=Vmax is represented as VmaxT. 20 Xₖ(that is, k is an integer ranging from 1 to 20) are set, and are each defined as a value gradually increasing from Vmax×3% (k=1) to Vmax×98% (k=20) at regular intervals (5% increments). Lines each indicating Xₖ are drawn below the curve of V. For each Xₖ, there are two time points at which V is Xₖ (intersections between the line of Xₖ and V). One is present before VmaxT, and the other is present after VmaxT. As a result, for 20 Xₖ, 40 time points at which V=Xₖ is satisfied are detected. Among these time points, 20 items (from t[1] to t[20]) before VmaxT correspond to pₖ, and 20 items (from t[40] to t[21]) after VmaxT correspond to qₖ.

Based on the pₖ and qₖ described above, the parameter for analyzing the blood coagulation ability of the test specimen can be calculated. An example of the parameter may be the difference of averages of pₖ and qₖ (hereinafter also called the pre-post average difference). In the present Description, the average of point pₖ present before V reaches Vmax is sometimes called pre-Ave. Likewise, the average of point qₖ present after V reaches Vmax is sometimes called post-Ave, and the average of all pₖ and qₖ is sometimes called all-Ave. For example, pre-Ave, post-Ave, and all-Ave can be calculated from all pₖ (pₗ, . . . , pₙ) and all qₖ(qₗ, . . . , qₙ) (n is as defined above). Alternatively, pre-Ave, post-Ave, and all-Ave can be calculated as averages of pₖ and qₖ in any range (for example, (pₐ, . . . , p_{b}) and (qₐ, . . . , q_{b}), where a and b are integers independently selected from the group consisting of 1 to n, a<b, and n is as defined above). In one embodiment, the pre-post average difference is relativize, and is defined as, for example, the ratio of the difference between pre-Ave and post-Ave to all-Ave ([(post-Ave−pre-Ave)/all-Ave]).

Another example of the parameter may be the coefficient of variation of pₖ and qₖ (hereinafter, also called CV). For example, CV can be defined as the coefficient of variation (%) of all pₖ (pₗ, . . . , pₙ) and all qₖ (qₗ, . . . , qₙ) (n is as defined above). Alternatively, CV can be defined as the coefficient of variation (%) of pₖ and qₖ in any range (for example, (pₐ, . . . , p_{b}) and (qₐ, . . . , q_{b}), where a and b are integers independently selected from the group consisting of 1 to n, a<b, and n is as defined above). As described in after-mentioned Example, the size of the peak width of V of the specimen containing the FVIII-substituting substance, or the asymmetric degree of the peak shape is reflected in the pre-post average difference and CV.

Another example of the parameter may be the ratio of qₖ and pₖ (that is, qₖ/pₖ; hereinafter, also called the qp ratio) (k is any integer selected from the group consisting of 1 to n, and n is as defined above). Another example of the parameter may be a reference length VB(Sₖ), which represents an interval from pₖ to qₖ (time length or the number of measurement points) (k denotes an integer selected from the group consisting of 1 to n, and n is as defined above). In a case where V is an unimodal peak shape as illustrated in FIG. 2, VB(Sₖ) corresponds to an interval from pₖ to qₖ (=[qₖ−pₖ], or =[qₖ−pₖ+D], D is defined as described above) As described in after-mentioned Example, the size of the peak width of V of the specimen containing the FVIII-substituting substance, or the asymmetric degree of the peak shape is reflected also in the qp ratio and VB(Sₖ).

Another example of the parameter may be parameters about the relative values of pₖ and qₖ. Hereinafter, the relative values of pₖ and qₖ are respectively called a relative pₖ (or rpₖ) and a relative qₖ (or rqₖ). In one embodiment, rpₖ=pₖ/C and rqₖ=qₖ/C, and rpₖ (rpₐ, . . . , rp_{b}) with k being in a range from a to b and/or rqₖ (rq_{c}, . . . , rq_{d}) with k being in a range from c to d (here, a, b, c, and d are integers independently selected from the group consisting of 1 to n, a<b, c<d, and n is as defined above) are obtained. Here, C is a function with respect to time or the number of measurement points, and may be, for example, VmaxT, APTT or an equivalent in the number of measurement points, any pₖ and qₖ, or an after-mentioned weighted average time VT(Sₖ). A value in which the difference between the obtained (rpₐ, . . . , rp_{b}) and/or (rqₐ, . . . , rq_{d}), and (rpₐ, . . . , rp_{b}) and/or (rq_{c}, . . . , rq_{d}) of the reference specimen is reflected is adopted as the parameter. For example, the slope 6 of the linear regression line of a plot of (rpₐ, . . . , rp_{b}) and/or (rq_{c}, . . . , rq_{d}) of the test specimen against (rpₐ, . . . , rp_{b}) and/or (rq_{c}, . . . , rq_{d}) of the reference specimen can be adopted as the parameter. The parameter is obtained by quantification which allows the degree of the difference in the progression of the coagulation reaction of the specimen containing the FVIII-substituting substance to be exhibited, using pₖ and qₖ which represent the progression of the coagulation reaction (coagulation reaction rate curve), with reference to the progression of the specimen containing no FVIII-substituting substance. The size of the peak width of V of the specimen containing the FVIII-substituting substance, or the asymmetric degree of the peak shape is reflected in this parameter.

As described above, the blood coagulation ability of the test specimen is reflected in the aforementioned pₖ, qₖ, or parameters calculated therefrom, which suggests presence of the FVIII-substituting substance in the test specimen. Using these parameters can detect the presence or absence of the FVIII-substituting substance in the test specimen.

### 4.3) Parameter Based on Weighted Average of V

In one embodiment, the parameter for analyzing the blood coagulation ability of the test specimen is a parameter based on the weighted average of V.

Specifically, they may be the kurtosis (vAB(Sₖ)) in a predetermined region below V(i) (i denotes time or the number of measurement points) and the rate over time (vTB(Sₖ)) of the following equation.

vAB(Sₖ)=vH(Sₖ)/vB(Sₖ)

vTB(Sₖ)=vB(Sₖ)/VmaxT

where

$$vH ( Sk ) = \frac{{\sum }_{i = pk}^{qk} ( i \times V ( i ) )}{{\sum }_{i = pk}^{qk} i}$$

pₖ, qₖ, Sₖ, and vB(Sₖ) are as defined in the aforementioned 4.2).

### That is,

V(pₖ−D)<Vmax×Sₖ(%)≤V(pₖ), and

V(qₖ)≥Vmax×Sₖ(%)>V(qₖ+D),
where pₖ and qₖ represent time or the number of measurement points, pₖ≤VmaxT≤qₖ, and VmaxT satisfies V(VmaxT)=Vmax,
D denotes measurement interval,
0<Sₖ≤100, preferably, 0.5<Sₖ≤99, and more preferably, 5≤Sₖ≤50.
k denotes an integer ranging from 1 to n,
n denotes an integer ranging, preferably, from 5 to 50, and more preferably, from 10 to 30,
VB(Sₖ) denotes an interval from pₖ to qₖ (time or the number of measurement points).

Note that a parameter, such as APTT or the equivalent in the number of measurement points, or the weighted average time vT(Sₖ) described below, can be used as the denominator of an equation for calculating vTB(Sₖ), instead of VmaxT. Note that vT(Sₖ) can be obtained as a function of time or a function of the number of measurement points, depending on whether i indicates time or the number of measurement points.

$$vT ( Sk ) = \frac{{\sum }_{i = pk}^{qk} ( i \times V ( i ) )}{{\sum }_{i = pk}^{qk} V ( i )}$$

vH(Sₖ) corresponds to the Y-coordinate of the weighted average point of the region satisfying V(i)≥Vmax×Sₖ(%) below V(i). vAB(Sₖ) and vTB(Sₖ) respectively represent the kurtosis of the region and the rate over time.

### 4.4) Parameter Based on Relative Value of V

In one embodiment, the relativized maximum of the first derivative curve (hereinafter, corrected Vmax) is determined. For example, R is relativized such that the maximum is a predetermined value (for example, 100), and subsequently, the maximum of the first derivative curve obtained by the first derivative thereof is determined as the corrected Vmax. Alternatively, the corrected Vmax is also obtained by multiplying Vmax by the correction coefficient (=the maximum of predetermined value/R). The corrected Vmax may be used as the parameter for analyzing the blood coagulation ability of the test specimen.

### 4.5) Other Parameters

A value obtained by multiplying or dividing the aforementioned parameter by a value about the coagulation reaction time or the number of measurement points, for example, VmaxT or vT(Sₖ), or APTT or their equivalent in the number of measurement points, may be used as the parameter for analyzing the blood coagulation ability of the test specimen in the present invention. Such parameters include [corrected Vmax×VmaxT], [corrected Vmax×APTT], and [vAB(Sₖ)×VmaxT]. The parameter in the present invention may be a parameter about time or a parameter about the number of measurement points, depending on whether i, pₖ, and qₖ are time or the number of measurement points, and in a case of multiplying or dividing the parameter as described above, it is preferable that the parameter about time be multiplied or divided by a function of time, and the parameter about the number of measurement points be multiplied or divided by a function of the number of measurement points.

### 4.6) Standard Deviation Index (SDI)

A value obtained by converting the unit of each of the parameters described in aforementioned 4.2) to 4.5) into the standard deviation index (SDI) with respect to a reference value can be used as the parameter for analyzing the blood coagulation ability of the test specimen in the present invention. Specifically, SDI obtained by the following equation using the statistics of the specimen containing no FVIII-substituting substance (that is, a specimen which contains no FVIII-substituting substance and has no prolonged coagulation time; hereinafter also called a non-administered (ND) specimen) can be adopted as the parameter. More preferably, the absolute value of SDI (|SDI|) obtained by the following equation can be adopted as the parameter: |SDI|=|{(parameter value calculated from the test specimen)−(average of parameter values calculated from ND specimen group)}/(standard deviation of parameter values calculated from the ND specimen group)|.

### 5. Detection of Specimen Containing FVIII-Substituting Substance

As described in the after-mentioned Example, the values of the aforementioned parameters calculated in the present invention for analyzing the blood coagulation ability, pre-post average difference, all CV, qp ratio, vB(Sₖ), vAB(Sₖ), vTB(Sₖ), parameters about the relative values of pₖ and qₖ, corrected Vmax, their SDI and the like showed different tendencies between specimens containing the FVIII-substituting substance obtained from patients to whom the FVIII-substituting substance (emicizumab) had been administered, and specimens containing no FVIII-substituting substance. Based on these parameters, the specimen containing the FVIII-substituting substance can be detected.

In analysis of the blood coagulation ability by the present invention, examples of parameters used to detect the specimen containing the FVIII-substituting substance include at least one selected from the group consisting of the aforementioned pre-post average difference, all CV, qp ratio, vB(Sₖ), vAB(Sₖ), vTB(Sₖ), parameters about relative values of pₖ and qₖ, and corrected Vmax. The parameters used to detect the specimen containing the FVIII-substituting substance also include values obtained by multiplying or dividing the parameters by values about the coagulation reaction time or the number of measurement points, for example, VmaxT, APTT, or its equivalent in the number of measurement points, or vT(Sₖ), for example, [corrected Vmax×VmaxT], [corrected Vmax×APTT], [vAB(Sₖ)×VmaxT], and [vAB(Sₖ)×APTT]. Alternatively, the SDI of each of the aforementioned parameters may also be an example of the parameter used to detect the specimen containing the FVIII-substituting substance. In the analysis of the blood coagulation ability by the present invention, these parameters may be any one kind of them, or two or more kinds may be combined and used. Preferably, at least one selected from the group consisting of the pre-post average difference, all CV, qp ratio, and vTB(Sₖ), parameters about relative values of pₖ and qₖ, and SDI of any of these parameters may be used.

In the detection of the specimen containing the FVIII-substituting substance based on the parameter, for example, in the cases where the parameters are about the pre-post average difference, all CV, qp ratio, vB(Sₖ), vTB(Sₖ), and relative values of pₖ and qₖ and where the parameter calculated from the test specimen is greater or equal to a predetermined detection threshold (hereinafter, also simply called “threshold”) or greater than the threshold, the test specimen can be detected as the specimen containing the FVIII-substituting substance. For example, in the cases where the parameters are vAB(Sₖ), [vAB(Sₖ)×VmaxT], [vAB(Sₖ)×APTT], corrected Vmax, [corrected Vmax×VmaxT], and [corrected Vmax×APTT] and where the parameter calculated from the test specimen is less than or equal to the threshold, or less than the threshold, the test specimen can be detected as the specimen containing the FVIII-substituting substance.

A value which can distinguish the specimen containing the FVIII-substituting substance, and the specimen containing no FVIII-substituting substance (ND specimen) from each other can be set as the threshold. For example, in the cases where the parameters are about the pre-post average difference, all CV, qp ratio, vTB(Sₖ), and relative values of pₖ and qₖ, the maximum of the parameter calculated from the ND specimen group, or average+K×standard deviation (SD) (preferably, K ranges from 1.5 to 3.4) can be set as the threshold. On the other hand in the cases where the parameters are [vAB(Sₖ)×VmaxT], [vAB(Sₖ)×APTT], [corrected Vmax×VmaxT], and [corrected Vmax×APTT], the minimum of the parameter calculated from the non-administered specimen group, or average−K×standard deviation (SD) (preferably, K ranges from 1.3 to 2.3) can be set as the threshold.

The specimen containing the FVIII-substituting substance and the ND specimen can be distinguished from each other with reference to a two-dimensionally changing threshold (threshold line) instead of a threshold which has a fixed value. For example, if a value of the test specimen plotted on (APTT, vB(Sₖ)) two-dimensional plane is above the threshold line (for example, [vB(Sₖ)=a×APTT+b]; a and b are any coefficients), the test specimen can be detected as the specimen containing the FVIII-substituting substance. For example, if a value of the test specimen plotted on (APTT, vAB(Sₖ)) plane or (APTT, corrected Vmax) plane is below the threshold line (for example, [vAB(Sₖ)=a×APTT+b] or [corrected Vmax=a×APTT+b]; a and b are any coefficients), the test specimen can be detected as the specimen containing the FVIII-substituting substance. The threshold line can be obtained by, for example, a procedure as described in after-mentioned Example 1.

In the case where the parameter is the SDI of each parameter described above, the threshold can be set based on the SDI of the parameter preliminarily calculated from the specimen containing the FVIII-substituting substance group or the ND specimen group. For example, the threshold for |SDI| described above can be set as follows: what has a value closest to the distribution region of the ND specimen group is extracted from among the parameters calculated from the specimen containing the FVIII-substituting substance group, and its |SDI| is obtained by the aforementioned equation; meanwhile, what has a value closest to the distribution region of the FVIII-substituting substance group is extracted from among the parameters calculated from the ND specimen group, and its |SDI| is obtained by the aforementioned equation; and an intermediate value between the former |SDI| and the latter |SDI| can be set as the threshold (see after-mentioned Example 9). Alternatively, instead of the intermediate value, any value which can distinguish the distributions of the former |SDI| and the latter |SDI| from each other may be set as the threshold. If |SDI| calculated from the test specimen is greater than the threshold, the test specimen can be detected as the specimen containing the FVIII-substituting substance.

Two or more of the parameters can be used as the reference for the detection. In this case, a threshold (or a threshold line) is set for each parameter, and it is determined whether each parameter calculated from the test specimen satisfies the reference based on the corresponding threshold (or the threshold line). In one example, if all the two or more parameters satisfy the threshold, the test specimen is detected as the specimen containing the FVIII-substituting substance. In another example, if at least one of the two or more parameters satisfies the threshold, the test specimen is detected as the specimen containing the FVIII-substituting substance.

The specimen detected as the specimen containing the FVIII-substituting substance is a specimen derived from a patient with an abnormality of the blood coagulation ability to which the FVIII-substituting substance has been administered, and the patient is estimated as a patient with a high bleeding risk in surgery or the like. Clinically, emicizumab, which is a drug for suppressing the bleeding tendency of hemophilia A, has been known as an FVIII-substituting substance. Consequently, the specimen containing the FVIII-substituting substance detected by the method of the present invention can be estimated as a specimen derived from a patient with hemophilia A to whom emicizumab has been administered.

As for the aforementioned detection reference, those skilled in the art can understand that as long as the specimen containing the FVIII-substituting substance can be detected, references which are “greater than the threshold” and “the greater than or equal to the threshold” can be interchangeably used, and likewise, references which are “less than the threshold” and “less than or equal to the threshold” can be interchangeably used.

### 6. Application to Other Coagulation Reaction Measurement Methods

The method of the present invention has thus been described above using, as the example, the case of coagulation reaction measurement based on the scattered light intensity. However, those skilled in the art can apply other coagulation reaction measurement methods (for example, coagulation reaction measurement based on the transmittance, absorbance, viscosity and the like) to the coagulation reaction measurement in the present invention. Accordingly, such applications are encompassed by the scope of the present invention. For example, in the reaction R(i) represented by an inverted sigmoidal coagulation reaction curve based on the transmittance (transmittance light intensity), the positive and the negative are reversed with respect to that described based on the scattered light intensity. It is obvious to those skilled in the art that in such a case, in the aforementioned method of the present invention, the signs of R(i) and V(i) are reversed, the minimum Vmin is determined instead of the maximum Vmax of V, and pₖ and qₖ are respectively determined as points which reach Xₖ before or after V(i) reaches Vmin.

### 7. Program and Device

The method for analyzing the blood coagulation ability in the present invention can be automatically performed using a computer program. Consequently, an aspect of the present invention is a program for performing the aforementioned method for analyzing the blood coagulation ability in the present invention. A series of steps of the aforementioned method of the present invention can be automatically performed by an automatic analysis device. Consequently, an aspect of the present invention is a device for performing the aforementioned method for analyzing the blood coagulation ability in the present invention. Execution of the method of the present invention by the device can be controlled by the program in the present invention. Another aspect of the present invention is a system for performing the method for analyzing the blood coagulation ability in the present invention. The system includes the device or the program for performing the aforementioned method for analyzing the blood coagulation ability in the present invention.

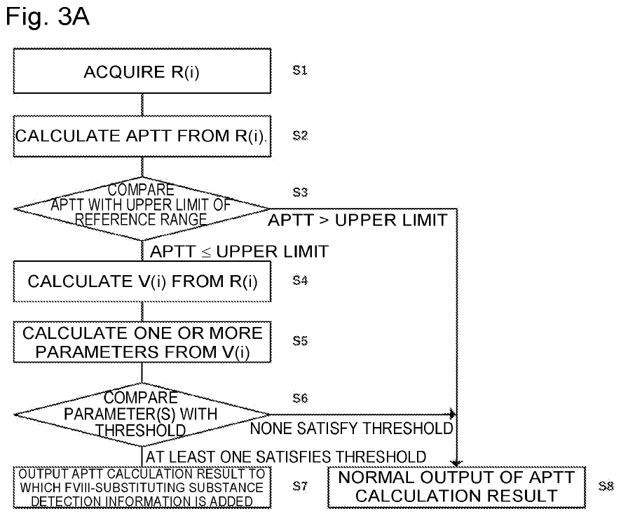

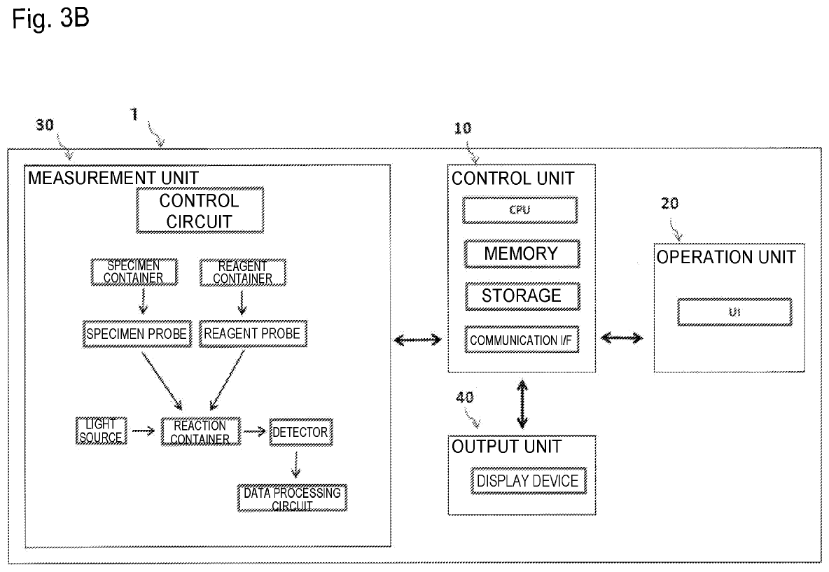

An example of a blood coagulation analyzing process by the present invention executed under control of the program of the present invention is described below and in FIG. 3A.
S1: Acquire R(i) from (freely) measured coagulation reaction data.
S2: Calculate APTT from R(i).
S3: Go to S4 if APTT is not prolonged, and to S8 if APTT is prolonged.
S4: Calculate V(i) from R(i).
S5: Calculate one or more parameters from V(i).
S6: Compare the parameter(s) with threshold.
   If at least one parameter satisfies the threshold, go to S7.
   If no parameter satisfies the threshold, go to S8.
S7: Output detection of APTT and the specimen containing the FVIII-substituting substance.
S8: Output APTT.

One embodiment of the device in the present invention is described below. The one embodiment of the device of the present invention is an automatic analysis device 1 as illustrated in FIG. 3B. The automatic analysis device 1 includes a control unit 10, an operation unit 20, a measurement unit 30, and an output unit 40.

The control unit 10 controls the entire operation of the automatic analysis device 1. The control unit 10 may be made up of a computer (for example, a personal computer) The control unit 10 includes a CPU, a memory, a storage, a communication interface (I/F) and the like, and can perform processing of commands from the operation unit 20, control of the operation of the measurement unit 30, storing and data analysis of measured data received from the measurement unit 30, storing of an analysis result, control of outputting of the analysis result by the output unit 40 and the like. Furthermore, the control unit 10 may be connected to other devices, such as an external medium, and a host computer. Note that in the control unit 10, the computer which controls the operation of the measurement unit 30, and the computer which analyzes data measured by the unit 30 may be identical to or different from each other.

The operation unit 20 acquires input from an operator, and transmits the obtained input information to the control unit 10. For example, the operation unit 20 includes an user interface (UI), such as a keyboard and a touch panel. Under control of the control unit 10, the output unit 40 outputs: a detection result of the specimen containing the FVIII-substituting substance, and coagulation reaction data measured by the measurement unit 30 as required; and R, V, and a parameter for analyzing the blood coagulation ability based thereon, and an analysis result, such as the coagulation time (APTT) as required. For example, the output unit 40 includes a display device, such as a display.

The measurement unit 30 executes the series of operations for analyzing the blood coagulation, and acquires measured data of the coagulation reaction of a sample which contains the blood specimen. The measurement unit 30 includes various instruments and analysis modules required to analyze the blood coagulation, for example, a specimen container which stores the blood specimen, a reagent container which stores a reagent for a test, a reaction container for the reaction between the specimen and the reagent, a probe for dispensing the blood specimen and the reagent into the reaction container, a light source, a detector for detecting scattered light or transmitted light from the sample in the reaction container, a data processing circuit which transmits data from the detector to the control unit 10, and a control circuit which controls the operation of the measurement unit 30 in response to an instruction of the control unit 10. Alternatively, in a case where V is acquired using known coagulation reaction data, and the parameter is calculated, the measurement unit 30 is not required.

The control unit 10 performs analysis of the coagulation reaction of the specimen, based on the coagulation reaction data. This analysis may include acquisition of the aforementioned R and V, calculation of APTT, calculation of various parameters, and detection of the specimen containing the FVIII-substituting substance using the parameter. Alternatively, R and V may be created by the control unit 10 based on the measured data from the measurement unit 30, or may be created by another device, for example, the measurement unit 30 and then be transmitted to the control unit 10. The control unit 10 may store a threshold used to detect the specimen containing the FVIII-substituting substance, or the control unit 10 may import the threshold stored in an external device or on a network, for the sake of the detection.

This analysis described above can be executed by the program for performing the method of the present invention. Consequently, the control unit 10 may include the program for performing the method for analyzing the blood coagulation ability in the present invention.

The analysis result in the control unit 10 is transmitted to the output unit 40 and is output. The output may be in any form, such as being displayed on a screen, being transmitted to a host computer, or being printed. Output information from the output unit can include R or V, APTT, a parameter for analyzing the blood coagulation ability, and a detection result of the specimen containing the FVIII-substituting substance. Furthermore, the output information may include other information obtained by APTT measurement (detection of an initial response, possibility of a chyle specimen and the like). The kind of output information from the output unit can be controlled by the program of the present invention.

### EXAMPLES

The present invention is described in further detail below with reference to Examples. However, the present invention is not limited to these Examples.

### 1. Method

### 1.1) Specimen

Control specimen group (NE): plasma (n=30) with APTT being within the reference standard range (from 24 to 39 seconds) of Coagpia APTT-N (manufactured by Sekisui Medical Co., Ltd.).
Test specimen group (E1): plasma (n=54) obtained from a patient with hemophilia A to whom emicizumab had been administered. The emicizumab concentration ranged from 12.2 to 79.2 μg/mL.
Test specimen group (E2): plasma (n=15) obtained from a patient with hemophilia A who had an antibody (inhibitor) against the coagulation factor VIII (FVIII) and to whom emicizumab had been administered. The emicizumab concentration ranged from 12.0 to 64.0 g/mL, and the inhibitor potency ranged from 0.8 to 202 BU/mL.

### 1.2) Coagulation Reaction Measurement

Coagpia APTT-N (manufactured by Sekisui Medical Co., Ltd.), which is a reagent for APTT measurement, was used as reagent for measurement, and Coagpia APTT-N calcium chloride solution (manufactured by Sekisui Medical Co., Ltd.) was used as a calcium chloride solution. The coagulation reaction of a sample containing a specimen was measured using the blood coagulation automatic analysis device CP3000 (manufactured by Sekisui Medical Co., Ltd.). 50 μL of the specimen was heated in a cuvette at 37 °C for 45 seconds, 50 μL of the reagent for measurement at approximately 37 °C was subsequently added, and after 171 seconds elapsed, 50 μL of the calcium chloride solution was added, thus initiating the coagulation reaction. The reaction was performed at 37 °C. In the measurement of the coagulation reaction, the cuvette was irradiated with light of a wavelength of 660 nm from a light source which is an LED, and the scattered light intensity of 90-degree side scattered light was measured at 0.1-second intervals. The measurement time was 360 seconds.

### 1.3) Acquisition of R(i) and V(i)

To measured data of the coagulation reaction from each specimen, after a smoothing process including denoising was applied, a zero adjustment process was applied so as to allow the scattered light intensity at the measurement initiation time point to be zero, and the coagulation reaction R(i) (i is time from the coagulation reaction initiation) was created. From R(i), the first derivative (coagulation reaction rate) V(i) was calculated. The maximum Vmax of V(i) of each specimen, and the time VmaxT at which V(i) reached the maximum Vmax were calculated.

### 1.4) APTT Calculation

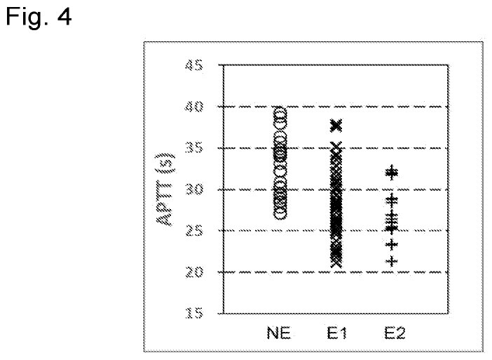

APTT of each specimen was calculated by the percent detection method. That is, a time point at which R(i) reached maximum Rmax within the measurement time was determined as a coagulation reaction end point E, and a time point at which R(i) reached 50% of R(E) was adopted as APTT. FIG. 4 illustrates APTT of NE, E1, and E2. APTT ranged from 27.1 to 39.3 seconds for NE, from 21.2 to 37.9 seconds for E1, and from 21.3 to 32.3 seconds for E2. The APTT distribution regions of E1 and E2 overlap the distribution region of NE. E1 and E2 exhibited APTT shorter than the lower limit (24 seconds) of the reference standard range in some cases. This result showed that the emicizumab-administered groups (E1 and E2), and the non-emicizumab-administered group (NE) cannot be distinguished from each other based only on APTT.

### 1.5) Creation of Corrected Reaction Rate Curve

To compare the shapes of rate curves of the specimens, a corrected reaction rate curve was created in the next procedure. The curve was relativized such that the maximum of V(i) was 100, and shifted in the temporal axis direction such that the time of reaching the maximum was 40 seconds, thus obtaining a corrected reaction rate curve. To display this corrected reaction rate curve, the abscissa axis name was defined as the corrected time, and the ordinate axis name was defined as the corrected reaction rate.

### 1.6) Calculation of Corrected Vmax

By multiplying Vmax of each specimen by a coefficient F (=100/Rmax), and the corrected Vmax (=Vmax×F) was calculated.

1.7) Calculation of Parameter about V(i)

The parameter about V(i) of each specimen was calculated. It was defined that Xₖ=Vmax×Sₖ(%) (k is an integer from 1 to 20, and Sₖ increases from 3% to 98% in increments of 5%; that is, S₁=3%, and S₂₀=98%). 20 pₖ (p₁, . . . , p₂₀) including minimums with i satisfying V(i)≥Xₖ with each k at time points before VmaxT, and 20 qₖ (q₁, . . . , q₂₀) including maximums with i satisfying V(i)≥Xₖ with each k at time points after VmaxT were acquired (see FIG. 2). From the obtained pₖ and qₖ, the following parameters were calculated.
Pre-Ave: the average of pₖ (k is any integers selected from a range from 1 to 20),
Post-Ave: the average of qₖ (k is as described above),
All-Ave: the average of pₖ and qₖ,
Pre-post average difference: (post-Ave−pre-Ave)/all-Ave,
CV: coefficient of variation (%) about pₗ and qₖ, qp ratio: qₖ/pₖ,
kurtosis vAB (Sₖ)=vH (Sₖ)/vB (Sₖ), or

vAB(Sₖ)=vH(Sₖ)/vB(Sₖ)×VmaxT, or

vAB(Sₖ)=vH(Sₖ)/vB(S_{K})×APTT,
Rate over time vTB(Sₖ)=vB (Sₖ)/VmaxT.

Note that vH(Sₖ) and vB(Sₖ) are as follows
vB(Sₖ): qₖ−pₖ+0.1 (seconds)

$$vH ( Sk ) = \frac{{\sum }_{i = pk}^{qk} ( i \times V ( i ) )}{{\sum }_{i = pk}^{qk} i}$$

(pₖ, qₖ, Sₖ, and k are as described above)

Hereinafter, parentheses on both the sides in each parameter name are sometimes abbreviated for representation. For example, vB(30) is sometimes represented as vB30.

### 2. Coagulation Reaction

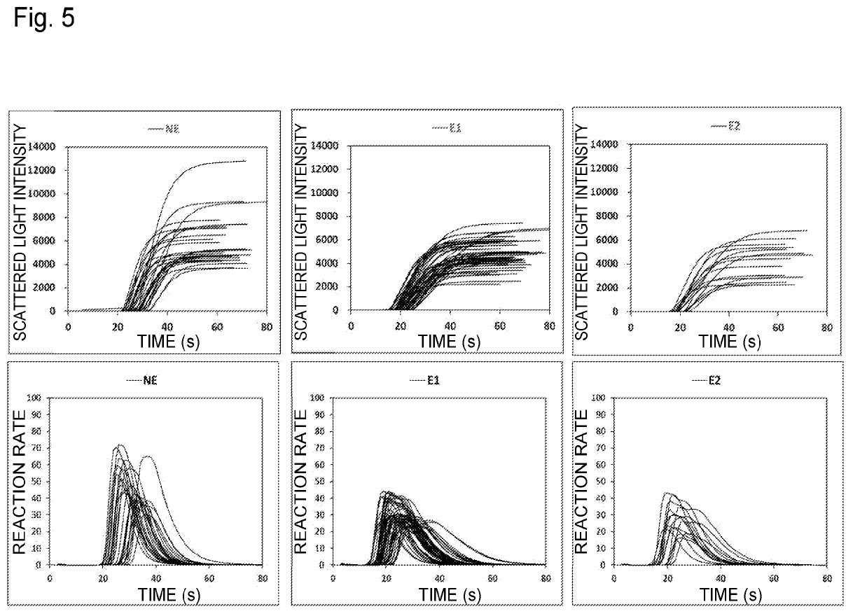

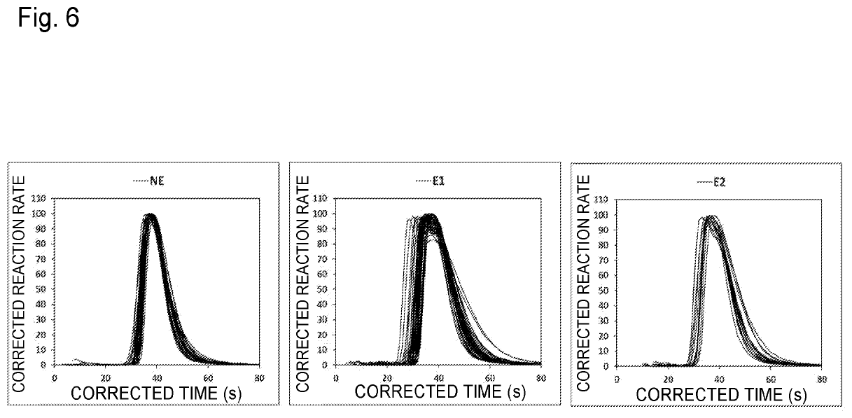

FIG. 5 illustrates the coagulation reaction curves R(i) (zero-adjusted scattered light intensity) (upper), and the first derivative curve V(i) (lower) of NE, E1, and E2 plotted against the measurement time. E1 and E2, in comparison with NE, had tendencies where the rise time of the reaction curve was rapid but the increase of the curve was gradual. FIG. 6 illustrates the corrected reaction rate curves of NE, E1, and E2 plotted against the corrected time. In comparison with NE, the corrected reaction rate curves of E1 and E2 had tendencies where the peak width was wide, and the peak shape was strongly asymmetric (the descent from the peak top was gradual).

### 3. Comparison of Parameters Between Specimen Groups

### 3.1) Comparative Example 1 Vmax

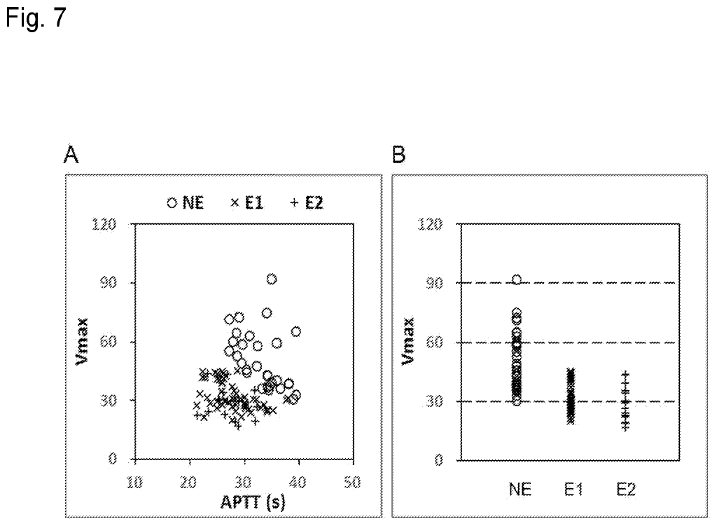

FIG. 7 illustrates a distribution of the maximums Vmax of V(i) about NE, E1, and E2. FIG. 7A illustrates a plot of Vmax against APTT of each specimen. FIG. 7B illustrates a Vmax distribution of each specimen group. Vmax of E1 and E2 tended to be smaller than that of NE, but the distribution regions overlapped that of NE. The emicizumab-administered groups (E1 and E2), and non-administered group (NE) were unable to be distinguished from each other based only on Vmax.

### 3.2) Comparative Example 2 VmaxT

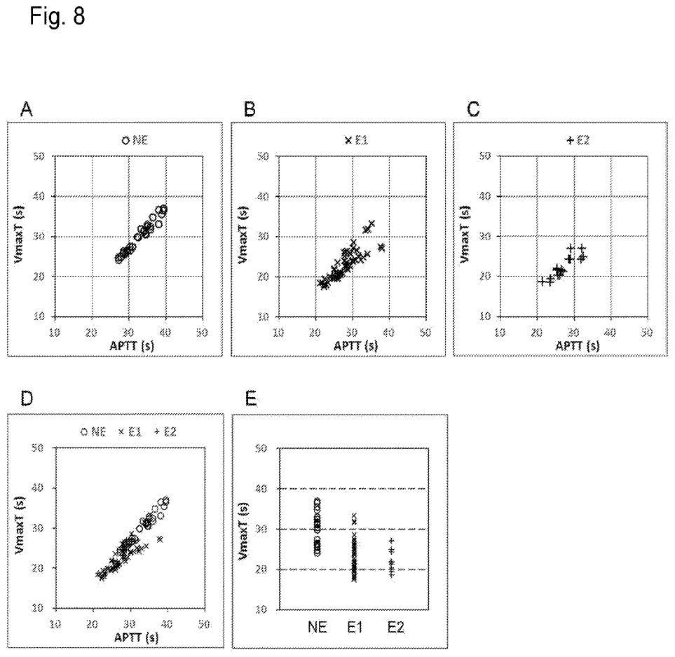

FIGS. 8A to 8D illustrate a plot of VmaxT against APTT about NE, E1, E2, and all specimens, respectively. FIG. 8E illustrates the VmaxT distribution for each specimen group. While VmaxT of E1 and E2 tended to be smaller than that of NE, the distribution regions overlapped that of NE. The emicizumab-administered groups (E1 and E2), and non-administered group (NE) were unable to be distinguished from each other based only on VmaxT.

### 3.3) Example 1 Corrected Vmax

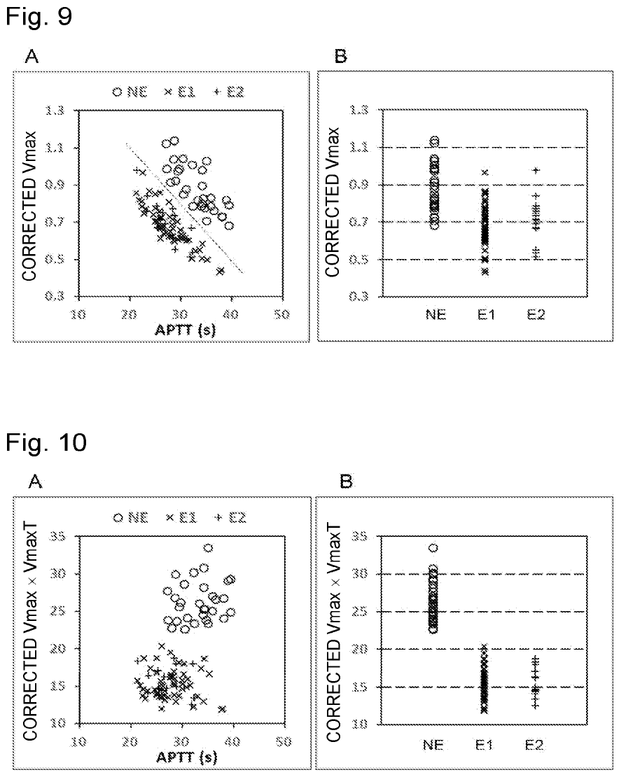

FIG. 9 illustrates a corrected Vmax distributions for NE, E1, and E2. FIG. 9A illustrates a plot of Vmax against APTT of each specimen. FIG. 9B illustrates the corrected Vmax distribution of each specimen group. The dotted line in FIG. 9A was obtained by the following procedure: 1) a regression line y=ax+b of the plot of the specimens of NE was obtained; 2) a plotted point (outermost point) which was closer to distributions of E1 and E2 than the regression line among the plotted points of NE, and was farthest in distance from the regression line was obtained; 3) a plotted point (closest point) which was closest to the regression line among the plotted points of E1 and E2 was obtained; and 4) A line which passed through the midpoint of a line connecting the outermost point and the closest point and was parallel to the regression line was calculated. It was shown that the distribution region of the corrected Vmax was different between the emicizumab-administered groups (E1 and E2) and the non-administered group (NE), and both the groups were able to be divided from each other by the dotted line of FIG. 9A, the dotted line obtained by the procedure can be used as a two-dimensional threshold (threshold line) distinguishing the emicizumab-administered groups (E1 and E2) and the non-administered group (NE). It was shown that by setting such a threshold line, the emicizumab-administered groups (E1 and E2) and the non-administered group (NE) could be distinguished from each other based on APTT and the corrected Vmax.

FIG. 10A shows plots of [corrected Vmax×VmaxT] of the specimens of NE, E1, and E2 against APTT. FIG. 10B illustrates a distribution of [corrected Vmax×VmaxT] of each specimen group. It was shown that the distribution region of [corrected Vmax×VmaxT] was different between the emicizumab-administered groups (E1 and E2) and the non-administered group (NE), and the emicizumab-administered groups (E1 and E2) and the non-administered group (NE) could be distinguished from each other based on the value.

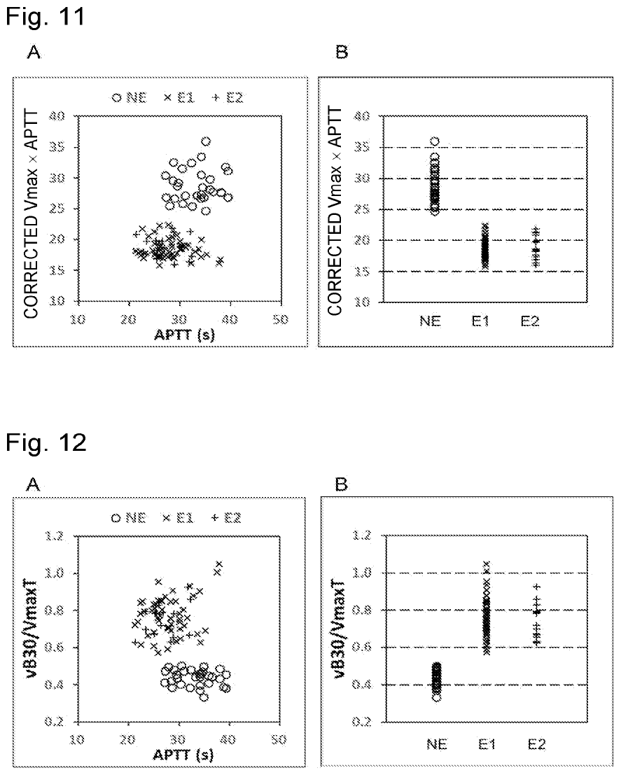

FIG. 11A illustrates plots of [corrected Vmax×APTT] of the specimens of NE, E1, and E2 against APTT. FIG. 11B illustrates a distribution of [corrected Vmax×APTT] of each specimen group. It was shown that the distribution region of [corrected Vmax×APTT] was different between the emicizumab-administered groups (E1 and E2) and the non-administered group (NE), and the emicizumab-administered groups (E1 and E2) and the non-administered group (NE) could be distinguished from each other based on the value. The results in FIGS. 10 and 11 showed that using the value obtained by multiplying the corrected Vmax by the parameter about the reaction time, such as VmaxT or APTT, could distinguish the emicizumab-administered groups (E1 and E2) and the non-administered group (NE) from each other with reference to a specific threshold without the need to set a two-dimensional threshold (threshold line).

### 3.4) Example 2 VTB(Sₖ)

FIG. 12A illustrates plots of vTB(30) of the specimens of NE, E1, and E2 against APTT. FIG. 12B illustrates a distribution of vTB(30) of each specimen group. In the diagram, vTB(30) is represented by vB(30)/VmaxT. It was shown that the distribution region of vTB(30) was different between the emicizumab-administered groups (E1 and E2) and the non-administered group (NE), and the emicizumab-administered groups (E1 and E2) and the non-administered group (NE) could be distinguished from each other based on the value.

### 3.5) Example 3 vAB(Sₖ)

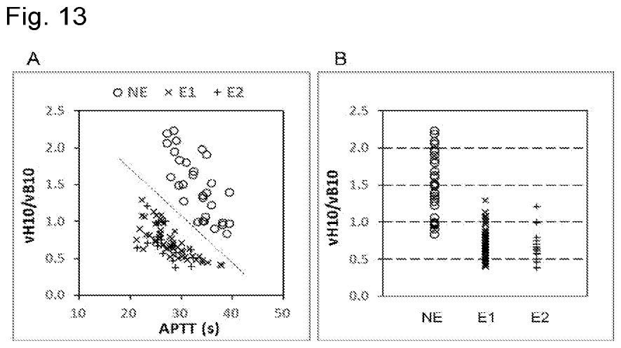

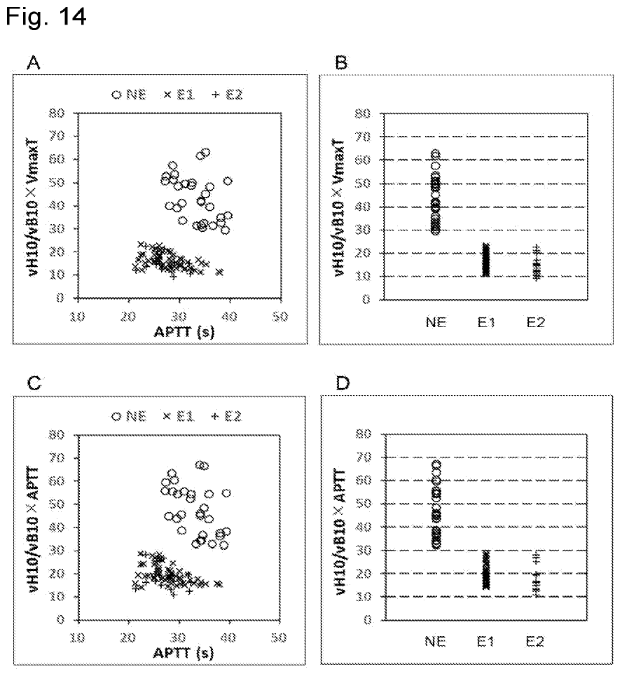

FIG. 13A illustrates plots of vAB(10)=vH(10)/vB(10) of the specimens of NE, E1, and E2 against APTT. FIG. 13B illustrates a distribution of vAB(10) of each specimen group. In the diagram, vAB(10) is represented by vH(10)/vB(10). The dotted line in FIG. 13A was calculated in a manner similar to that of Example 1 (3.3 described above). The distribution region of vAB(10) was different between the emicizumab-administered groups (E1 and E2) and the non-administered group (NE). The groups could be divided by the dotted line in FIG. 13A. It was shown that by setting such a two-dimensional threshold (threshold line), the emicizumab-administered groups (E1 and E2) and the non-administered group (NE) could be distinguished from each other based on APTT and the vAB(Sₖ).

FIG. 14A shows plots of [vAB(10)×VmaxT] of the specimens of NE, E1, and E2 against APTT. FIG. 14B illustrates a distribution of [vAB(10)×VmaxT] of each specimen group. FIG. 14C shows plots of [vAB(10)×APTT] of the specimens of NE, E1, and E2 against APTT. FIG. 14D illustrates a distribution of [vAB(10)×APTT] of each specimen group. In the diagram, vAB(10) is represented by vH(10)/vB(10). [vAB(10)×VmaxT] and [vAB(10)×APTT] of the emicizumab-administered groups (E1 and E2) were lower than those of the non-administered group (NE). It was shown that using the value obtained by multiplying VAB(Sₖ) by VmaxT or APTT could distinguish the emicizumab-administered groups (E1 and E2) and the non-administered group (NE) from each other with reference to a specific threshold without the need to set a two-dimensional threshold (threshold line).

### 3.6) Example 4 qp Ratio

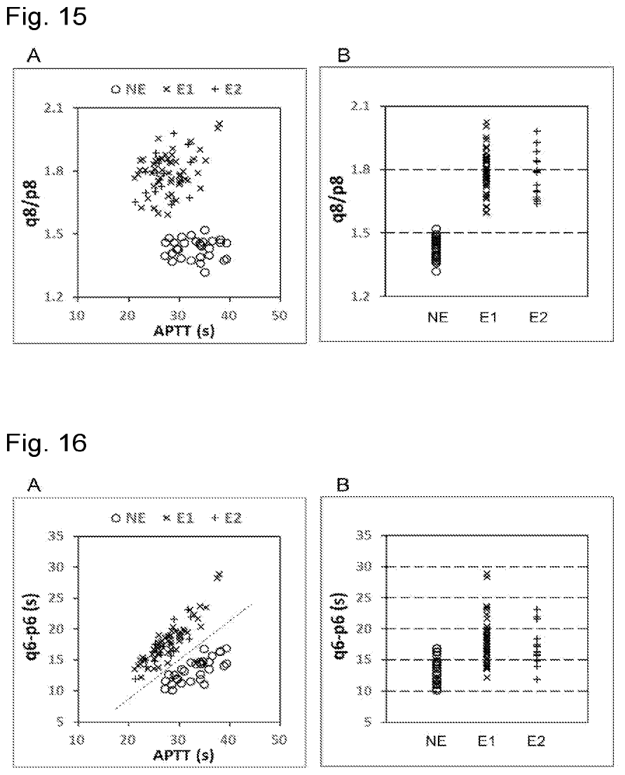

pₖ and qₖ with k=8 were calculated, and qp ratio=qₖ/pₖ was obtained. FIG. 15A illustrates plots of qp ratio (q₈/p₈) of the specimens of NE, E1, and E2 against APTT. FIG. 15B illustrates a distribution of qp ratio of each specimen group. It was shown that the distribution region of the pq ratio was different between the emicizumab-administered groups (E1 and E2) and the non-administered group (NE), and the emicizumab-administered groups (E1 and E2) and the non-administered group (NE) could be distinguished from each other based on the value. The size of the peak width of V(i) and the peak shape (asymmetric degree of the peak) in the emicizumab-administered groups (E1 and E2) against the non-administered group (NE) as shown in FIGS. 5 and 6 were estimated to be reflected in the distribution difference of the qp ratio between the emicizumab-administered groups (E1 and E2) and the non-administered group (NE).

### 3.7) Example 5 vB(Sₖ)

FIG. 16A illustrates plots of q₆−p₆ (vB(6)) of the specimens of NE, E1, and E2 against APTT. FIG. 16B illustrates a distribution of q₆−p₆ of each specimen group. The dotted line in FIG. 16A was calculated in a manner similar to that of Example 1 (3.3 described above). The distribution region of q₆−p₆ was different between the emicizumab-administered groups (E1 and E2) and the non-administered group (NE). The groups could be divided by the dotted line in FIG. 16A. It was shown that by setting such a two-dimensional threshold (threshold line) like the dotted line, the emicizumab-administered groups (E1 and E2) and the non-administered group (NE) could be distinguished from each other based on APTT and qₖ−pₖ (vB(Sₖ)). The size of the peak width of V(i) in the emicizumab-administered groups (E1 and E2) against the non-administered group (NE) as shown in FIGS. 5 and 6 were estimated to be reflected in the distribution difference of vB(Sₖ) between the emicizumab-administered groups (E1 and E2) and the non-administered group (NE).

### 3.8) Example 6 Pre-Post Average Difference

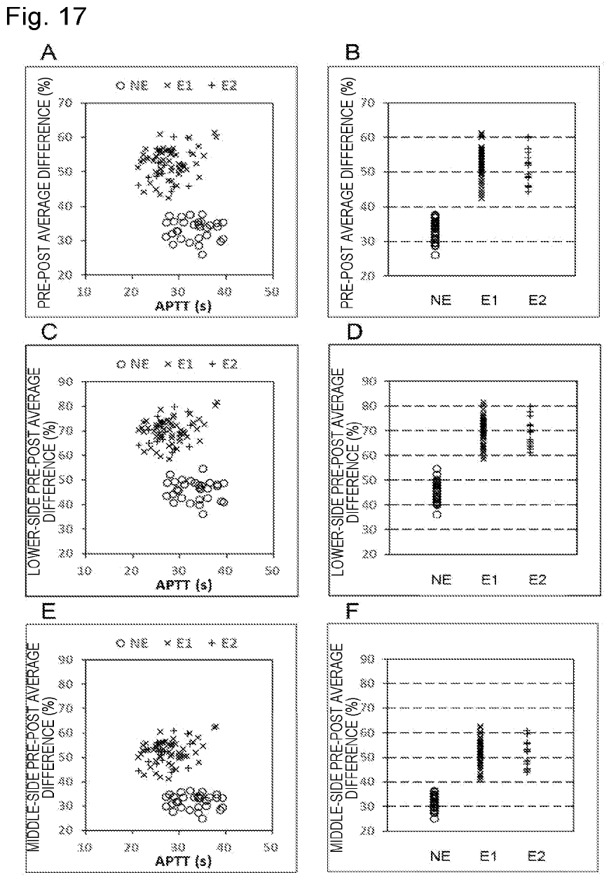

FIGS. 17A to 17F illustrate pre-post average difference of the specimens of NE, E1, and E2. FIGS. 17A, 17C, and 17E illustrate plots of the pre-post average difference against APTT. FIGS. 17B, 17D, and 17F illustrate a distribution of the pre-post average difference of each specimen group. FIGS. 17A and 17B illustrate the pre-post average difference about pₖ and qₖ detected at k=1 to 20, that is, from the entire peak of V. FIGS. 17C and 17D illustrate the pre-post average difference (lower-side pre-post average difference) about pₖ and qₖ detected at k=1 to 10, that is, from the lower side of the peak of V (lower-side pre-post average difference). FIGS. 17E and 17F illustrate the pre-post average difference about pₖ and qₖ detected at k=6 to 15, that is, from the middle of the peak of V (middle-side pre-post average difference). In all the cases, it was shown that the distribution region of the pre-post average difference was different between the emicizumab-administered groups (E1 and E2) and the non-administered group (NE), and the emicizumab-administered groups (E1 and E2) and the non-administered group (NE) could be distinguished from each other based on the pre-post average difference. The peak shapes of V(i) in the emicizumab-administered groups (E1 and E2) against the non-administered group (NE) (asymmetric degree of the peak) as shown in FIGS. 5 and 6 were estimated to be reflected in the distribution difference of the pre-post average difference between the emicizumab-administered groups (E1 and E2) and the non-administered group (NE).

### 3.9) Example 7 all CV

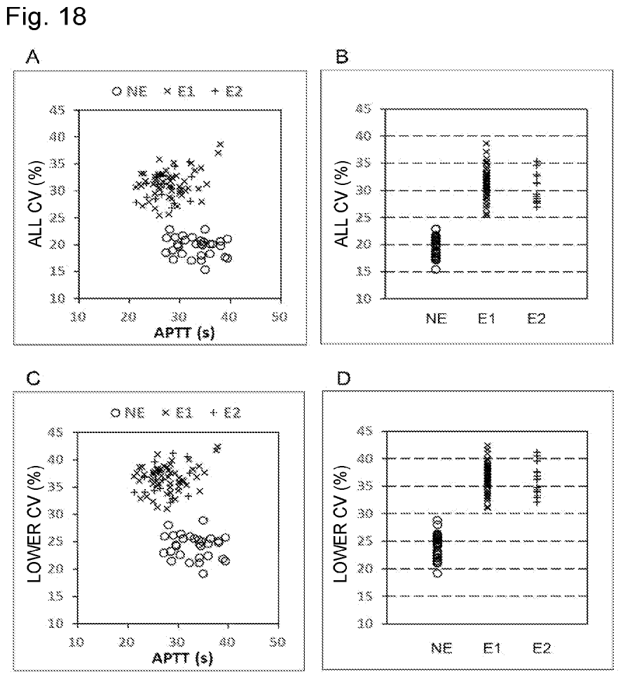

FIGS. 18A to 18D illustrate the CV of the pₖ and qₖ of the specimens of NE, E1, and E2. FIGS. 18A and 18C illustrate plots of CV against APTT. FIGS. 18B and 18D illustrate a distribution of CV of each specimen group. FIGS. 18A and 18B illustrate CV with p₁ to p₂₀ and q₁ to q₂₀ at k=1 to 20 (all CV). FIGS. 18C and 18D illustrate CV with p₁ to p₁₀ and q₁ to q₁₀ at k=1 to 10 (lower CV). It was shown that the distribution region of the CV of pₖ and qₖ was different between the emicizumab-administered groups (E1 and E2) and the non-administered group (NE), and the emicizumab-administered groups (E1 and E2) and the non-administered group (NE) could be distinguished from each other based on the CV of pₖ and qₖ. The size of the peak width of V(i) in the emicizumab-administered groups (E1 and E2), and the peak shape (asymmetric degree of the peak) as shown in FIGS. 5 and 6 were estimated to be reflected in the distribution difference of the CV of pₖ and qₖ between the emicizumab-administered groups (E1 and E2) and the non-administered group (NE).

### 3.10) Example 8 Ratio of pₖ and qₖ Against VmaxT

The pₖ and qₖ of NE, E1, and E2 were compared. The pₖ and qₖ of the specimens of NE, E1, and E2 (k=1 to 20 or k=6 to 20) were divided by VmaxT, and the relative pₖ (=pₖ/VmaxT) and relative qₖ (=qₖ/VmaxT) were obtained. The NE specimen with APTT at the minimum (27.1 seconds) or the maximum (39.3 seconds) was used as the reference specimen. A linear regression line was obtained between the relative pₖ and relative qₖ data groups of the reference specimen, and the relative pₖ and relative qₖ data groups of each specimen.

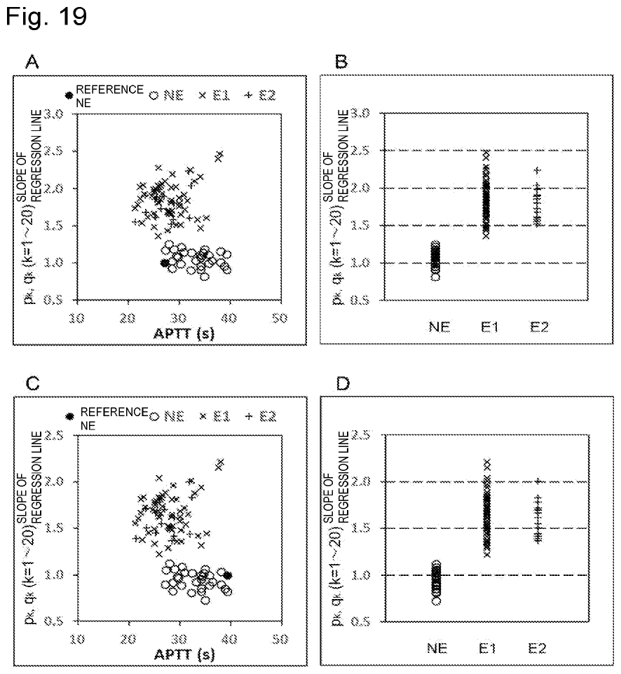

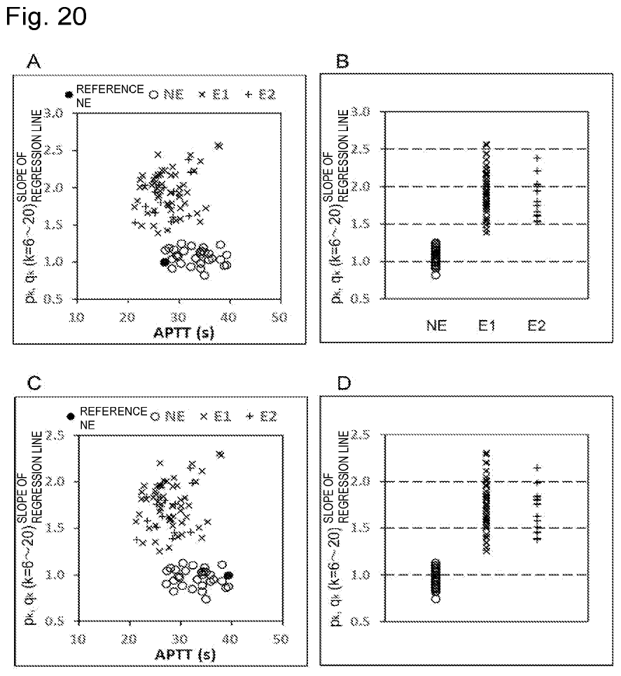

FIGS. 19A to 19D and FIGS. 20A to 20D illustrate distributions of the slopes of the regression lines of the specimens of NE, E1, and E2. FIG. 19 illustrates the slope of the regression line of the relative pₖ and the relative qₖ (k=1 to 20). FIG. 20 illustrates the slope of the regression line of the relative pₖ and the relative qₖ (k=6 to 20). As the reference specimen, the NE specimen with the minimum APTT was used in FIGS. 19A, 19B, 20A, and 20B, and the NE specimen with the maximum APTT was used in FIGS. 19C, 19D, 20C, and 20D. FIGS. 19A, 19C, 20A, and 20C illustrate plots of the slopes of the specimen against APTT. FIGS. 19B, 19D, 20B, and 20D illustrate a slope distribution of each specimen group. In FIGS. 19A, 19C, 20A, and 20C, the plotted points of the reference specimen (reference NE) are represented as solid circles ●. In all the cases in FIGS. 19 to 20, the slopes of the regression lines of the emicizumab-administered groups (E1 and E2) tend to be greater than that of the non-administered group (NE). They showed that the emicizumab-administered groups and the non-administered group could be distinguished from each other based on this parameter.

### 3.11) Example 9 SDI of Parameter

The proximity of the parameter (P) between the emicizumab-administered groups (Emi; including E1 and E2) and the non-administered group (NE) was evaluated. [corrected Vmax×VmaxT] illustrated in FIG. 10, [corrected Vmax×APTT] illustrated in FIG. 11, vTB(30) illustrated in FIG. 12, [vAB(10)×VmaxT] and [vAB(10)×APTT] illustrated in FIG. 14, q₈/p₈ illustrated in FIG. 15, the pre-post average difference, lower-side pre-post average difference, and middle-side pre-post average difference illustrated in FIG. 17, all CV and lower CV illustrated in FIG. 18, and four regression line slopes respectively illustrated in FIGS. 19A and 19B, FIGS. 19C and 19D, FIGS. 20A and 20B, and FIGS. 20C and 20D were used as the parameter (P). The specimen with P being closest to Emi in NE (that is, P was the maximum or minimum) (NE proximal specimen), and the specimen with P being closest to NE in Emi (that is, P was the minimum or maximum) (Emi proximal specimen) were extracted. The absolute value of the standard deviation index (|SDI|) of P of each of the extracted two specimens with respect to the average of P in NE was calculated.

|SDI|=|{(NE proximal specimen or P of Emi proximal specimen)−(average of P in NE)}/(standard deviation of P in NE)|

Table 1 illustrates |SDI| of the NE proximal specimen and the Emi proximal specimen of each parameter. In the difference of |SDI| between the NE proximal specimen and the Emi proximal specimen, the fact that each parameter had a different distribution between Emi and NE was reflected, as illustrated in Examples 1 to 8. These results showed that using |SDI| of the parameter illustrated in Examples 1 to 8 could distinguish the emicizumab-administered group and the non-administered group from each other. For example, |SDI| of the parameter of the test specimen with respect to the reference value was obtained, and was compared with the threshold, thereby allowing detecting whether the test specimen is in the emicizumab-administered group or not. The average of the parameters of NE as described above can be used as the reference value. An intermediate value between |SDI| of the NE proximal specimen and |SDI| of the Emi proximal specimen as described above can be set as an example of the threshold.

TABLE 1

| | | A: \|SDI\| of P of | B: \|SDI\| of P of | | Intermediate |
|---|---|---|---|---|---|
| | | NE proximal | Emi proximal | | value between |
| Drawings | Parameter (P) | specimen | specimen | B − A | A and B |
| FIG. 10A | Corrected Vmax × | 1.36 | 2.22 | 0.86 | 1.79 |
| | VmaxT | | | | |
| FIG. 11A | Corrected Vmax × APTT | 1.53 | 2.38 | 0.86 | 1.96 |
| FIG. 12A | vTB(30) | 1.47 | 3.15 | 1.68 | 2.31 |
| FIG. 14A | vAB(10) × VmaxT | 1.42 | 2.07 | 0.65 | 1.74 |
| FIG. 14C | vAB(10) × APTT | 1.42 | 1.76 | 0.33 | 1.59 |
| FIG. 15A | q8/p8 | 1.88 | 3.45 | 1.57 | 2.66 |
| FIG. 17A | Pre-post average | 1.52 | 3.06 | 1.54 | 2.29 |
| | difference | | | | |
| FIG. 17C | Lower-side pre-post | 2.10 | 3.04 | 0.94 | 2.57 |
| | average difference | | | | |
| FIG. 17E | Middle-side pre-post | 1.57 | 3.10 | 1.54 | 2.34 |
| | average difference | | | | |
| FIG. 18A | All CV | 1.76 | 3.17 | 1.41 | 2.47 |
| FIG. 18C | Lower CV | 2.13 | 3.10 | 0.97 | 2.61 |
| FIG. 19A | Regression line slope | 1.81 | 2.85 | 1.04 | 2.33 |
| FIG. 19C | Regression line slope | 1.78 | 2.88 | 1.10 | 2.33 |
| FIG. 20A | Regression line slope | 1.68 | 2.94 | 1.26 | 2.31 |
| FIG. 20C | Regression line slope | 1.67 | 2.95 | 1.28 | 2.31 |

1. A method for analyzing a blood coagulation ability of a blood specimen, the method comprising:
1) acquiring V(i), which is a first derivative of a coagulation reaction curve of a subject blood specimen in which presence or absence of a substance having coagulation factor VIII-substituting activity is required to be detected, where i denotes the number of measurement points or time; and
2) at least one of:
a) determining pₖ, which is a minimum of a point at which V(i) reaches Xₖ or higher when V(i) reaches a maximum Vmax or therebefore, and qₖ, which is a maximum of a point at which V(i) reaches Xₖ or higher when V(i) reaches the maximum Vmax or thereafter, where k represents a series of integers from 1 to n, n is an integer greater than or equal to two, Xₖ is Vmax×Sₖ%, and 0<Sₖ≤100;
b) determining a relativized maximum of V(i).

2. The method according to claim 1, wherein the subject blood specimen is a blood specimen with no prolonged activated partial thromboplastin time (APTT).

3. The method according to claim 1, wherein the n is an integer from 5 to 50.

4. The method according to claim 1, further comprising calculating a parameter for analyzing the blood coagulation ability of the subject blood specimen, based on the pₖ or qₖ.

5. The method according to claim 4,
wherein the parameter is at least one selected from the group consisting of a pre-post average difference, CV, qp ratio, vB(Sₖ), kurtosis vAB(Sₖ), rate over time vTB(Sₖ), vAB(Sₖ)×VmaxT, vAB(Sₖ)×APTT, parameters about relative values of pₖ and qₖ, and their standard deviation indices (SDI), where
the pre-post average difference represents (average of qₐ to q_{b}−average of pₐ to p_{b})/(average of pₐ to p_{b} and qₐ to q_{b}) (where a and b are integers selected from the group consisting of 1 to n, n is as defined above, and a<b),
the CV represents a coefficient of variation of pₐ to p_{b} and qₐ to q_{b} (where a and b are integers selected from the group consisting of 1 to n, n is as defined above, and a<b),
the qp ratio represents qₖ/pₖ,
the vB(Sₖ) represents an interval from pₖ to qₖ,
the VmaxT satisfies V(VmaxT)=Vmax,
the APTT is an activated partial thromboplastin time (APTT) of the subject blood specimen,
the vAB(Sₖ)=vH(Sₖ)/vB(Sₖ), and
the vTB(Sₖ)=vB(Sₖ)/VmaxT,
where
the vH(Sₖ) is of the following equation,
$$vH ( Sk ) = \frac{{\sum }_{i = pk}^{qk} ( i \times V ( i ) )}{{\sum }_{i = pk}^{qk} i}$$

6. The method according to claim 5,
wherein the parameters about the relative values of the pₖ and qₖ are slopes of linear regression lines of a plot from pₐ to p_{b} of the subject blood specimen and a plot from q_{c} to q_{d} of the subject blood specimen with respect to pₐ to p_{b} of a reference specimen and q_{c} to q_{d} of a reference specimen, respectively (where a, b, c, and d are integers each independently selected from the group consisting of 1 to n, a<b, c<d, and n is as defined above), and
the reference specimen is a blood specimen to which no drug having FVIII-substituting activity has been administered.

7. The method according to claim 1, further comprising calculating a parameter for analyzing the blood coagulation ability of the subject blood specimen, based on the relativized maximum of V(i).

8. The method according to claim 7,
wherein the parameter is at least one selected from the group consisting of corrected Vmax, corrected Vmax×VmaxT, corrected Vmax×APTT, and their standard deviation indices (SDI),
the corrected Vmax is the relativized maximum of V(i),
the VmaxT satisfies V(VmaxT)=Vmax, and
the APTT is an activated partial thromboplastin time (APTT) of the subject blood specimen.

9. The method according to claim 4, further comprising detecting the presence or absence of the substance having coagulation factor VIII-substituting activity in the subject blood specimen, based on the parameter.

10. The method according to claim 9, further comprising outputting a result of the detecting the presence or absence of the substance having coagulation factor VIII-substituting activity.

11. The method according to claim 1, further comprising:
measuring the activated partial thromboplastin time (APTT) of a blood specimen; and
selecting the blood specimen with no prolonged APTT, as the subject blood specimen in which the presence or absence of the substance having coagulation factor VIII-substituting activity is required to be detected.

12. The method according to claim 1, wherein the substance having coagulation factor VIII-substituting activity is a bispecific antibody substituting for a cofactor function of coagulation factor VIII.

13. The method according to claim 1, wherein the substance having coagulation factor VIII-substituting activity is emicizumab.

14. A program for implementing the method according to claim 1.

15. A device for implementing the method according to claim 1.

16. A system for implementing the method according to claim 1, the system comprising: a program for implementing the method according to claim 1; and a device controlled by the program.

17. The device according to claim 15, further comprising an output unit for outputting a result of analysis of the blood coagulation ability of the blood specimen by the method of:
1) acquiring V(i), which is a first derivative of a coagulation reaction curve of a subject blood specimen in which presence or absence of a substance having coagulation factor VIII-substituting activity is required to be detected, where i denotes the number of measurement points or time; and
2) at least one of (a) or (b):
a) determining pₖ, which is a minimum of a point at which V(i) reaches Xₖ or higher when V(i) reaches a maximum Vmax or therebefore, and qₖ, which is a maximum of a point at which V(i) reaches Xₖ or higher when V(i) reaches the maximum Vmax or thereafter, where k represents a series of integers from 1 to n, n is an integer greater than or equal to two, Xₖ is Vmax×Sₖ%, and 0<Sₖ≤100;
b) determining a relativized maximum of V(i).

18. The system of claim 16 further comprising an output unit for outputting a result of analysis of the blood coagulation ability of the blood specimen by the method comprising:
1) acquiring V(i), which is a first derivative of a coagulation reaction curve of a subject blood specimen in which presence or absence of a substance having coagulation factor VIII-substituting activity is required to be detected, where i denotes the number of measurement points or time; and
2) at least one of (a) or (b):
a) determining pₖ, which is a minimum of a point at which V(i) reaches Xₖ or higher when V(i) reaches a maximum Vmax or therebefore, and qₖ, which is a maximum of a point at which V(i) reaches Xₖ or higher when V(i) reaches the maximum Vmax or thereafter, where k represents a series of integers from 1 to n, n is an integer greater than or equal to two, Xₖ is Vmax×Sₖ%, and 0<Sₖ≤100;
b) determining a relativized maximum of V(i).
